(19) United States
(12) Patent Application Publication
(10) Pub. No.: US 2025/0107742 A1
(43) Pub. Date: Apr. 3, 2025

(54) TRAINING APPARATUS, METHOD, AND PROGRAM FOR NEUROFEEDBACK TRAINING

(71) Applicants: FrontAct Co., Ltd., Shinkawa, Chuo-ku, Tokyo (JP); Hiroshima University, Higashi-Hiroshima-shi, Hiroshima (JP)

(72) Inventors: Yasumasa OKAMOTO, Higashi-Hiroshima-shi, Hiroshima (JP); Go OKADA, Higashi-Hiroshima-shi, Hiroshima (JP); Naho ICHIKAWA, Higashi-Hiroshima-shi, Hiroshima (JP); Shigeyuki KAN, Higashi-Hiroshima-shi, Hiroshima (JP); Yuki MITSUYAMA, Higashi-Hiroshima-shi, Hiroshima (JP); Miyuki KATO, Higashi-Hiroshima-shi, Hiroshima (JP)

(21) Appl. No.: 18/833,191

(22) PCT Filed: Jan. 24, 2023
(86) PCT No.: PCT/JP2023/002081

§ 371 Date: Jul. 25, 2024

(30) Foreign Application Priority Data
Jan. 25, 2022 (JP) 2022-009579

(51) Int. Cl. A61B 5/375; A61B 5/00; A61B 5/374; A61B 5/378

(52) U.S. Cl. CPC A61B 5/375; A61B 5/374; A61B 5/378; A61B 5/742

(57) ABSTRACT

One or more embodiments of the invention is a training apparatus for neurofeedback training and includes an acquisition unit configured to acquire an electroencephalogram signal at a site in a left hemisphere of a subject and a control unit configured to cause a display device to display first teaching information for affecting an electroencephalogram signal for a predetermined time and to cause the display device to display second teaching information for affecting an electroencephalogram signal for a predetermined time after displaying the first teaching information. The control unit causes the display device to display result information generated based on at least one of an electroencephalogram signal received from the acquisition unit while displaying the first teaching information or an electroencephalogram signal received from the acquisition unit while displaying the second teaching information.

### TECHNICAL FIELD

The present invention relates to a training apparatus, method, and program for neurofeedback training.

### BACKGROUND ART

Neurofeedback methods have attracted attention as one of methods for treating depression (Non-Patent Documents 1 and 2) in recent years. For example, Patent Document 1 discloses a brain activity training apparatus for generating effective feedback information based on a correlation of specific connectivity of brain areas measured by a brain function imaging method such as MRI and performing training to change the correlation of the connectivity of the brain areas.

### CITATION LIST

### Patent Literature

Patent Document 1: Japanese Patent No. 6875054

### Non-Patent Literature

Non-Patent Document 1: Ichikawa Naho, Okamoto Yasumasa, “Current State and Prospects in Neurofeedback for Depression”, Japanese Journal of Molecular Psychiatry 14 (3), pp. 180-185, July 2014

Non-Patent Document 2: Takamura, M., Okamoto, Y., Shibasaki, C., et al., “Antidepressive Effect of Left Dorsolateral Prefrontal Cortex Neurofeedback in Patients with Major Depressive Disorder”, a preliminary report. Journal of Affective Disorder, 271: pp. 224-227, 2020

### SUMMARY OF INVENTION

### Technical Problem

Although training is performed while checking brain activity with, for example, functional MRI (fMRI) in the neurofeedback method of the related art, it has been desired to conduct training by using simpler equipment. The present invention has been made to solve this problem, and the main object is to provide a training apparatus and the like capable of more easily performing (or supporting) neurofeedback training.

### Solution to Problem

[1] A training apparatus according to an embodiment of the present invention is a training apparatus for neurofeedback training and includes an acquisition unit configured to acquire an electroencephalogram signal at a site in a left hemisphere of a subject and a control unit configured to cause a display device to display first teaching information for affecting an electroencephalogram signal for a predetermined time and to cause the display device to display second teaching information for affecting an electroencephalogram signal for a predetermined time after displaying the first teaching information. The control unit causes the display device to display result information generated based on at least one of an electroencephalogram signal received from the acquisition unit while displaying the first teaching information or an electroencephalogram signal received from the acquisition unit while displaying the second teaching information.
[2] In the training apparatus described in [1] according to an embodiment of the present invention, the first teaching information is information for prompting an increase in an intensity of the electroencephalogram signal acquired by the acquisition unit, and the second teaching information is information for prompting a decrease in the intensity of the electroencephalogram signal.
[3] In the training apparatus described in [1] or [2] according to an embodiment of the present invention, the control unit causes the display device to display first result information generated based on the electroencephalogram signal received from the acquisition unit while displaying the first teaching information after displaying the first teaching information and causes the display device to display second result information generated based on the electroencephalogram signal received from the acquisition unit while displaying the second teaching information after displaying the second teaching information, and the control unit performs display of the first teaching information, display of the first result information, display of the second teaching information, and display of the second result information in this order a predetermined number of times.
[4] In the training apparatus described in [3] according to an embodiment of the present invention, the control unit causes the display device to display information for resting for a predetermined time before displaying the first teaching information on the display device for training, determines a reference value based on an electroencephalogram signal received from the acquisition unit while the information for resting is being displayed, and updates the reference value based on the electroencephalogram signal received from the acquisition unit while the first teaching information is being displayed and the electroencephalogram signal received from the acquisition unit while the second teaching information is being displayed each time the display of the first teaching information, the display of the first result information, the display of the second teaching information, and the display of the second result information are performed in this order, and the control unit generates the first result information based on the electroencephalogram signal received from the acquisition unit while the first teaching information is being displayed and the reference value determined or updated and generates the second result information based on the electroencephalogram signal received from the acquisition unit while the second teaching information is being displayed and the reference value determined or updated.
[5] In the training apparatus described in [3] or [4] according to an embodiment of the present invention, the control unit performs the display of the first teaching information, the display of the first result information, the display of the second teaching information, and the display of the second result information in this order a predetermined number of times and then causes result information based on the first result information and the second result information to be displayed.
[6] In the training apparatus described in any of [1] to [5] according to an embodiment of the present invention, the acquisition unit acquires an electroencephalogram signal at an FC5 site of a subject according to the International 10-20 System or a site within a predetermined distance from the site.
[7] In the training apparatus described in any of [1] to [6] according to an embodiment of the present invention, the electroencephalogram signal received from the acquisition unit is an electroencephalogram signal in a θ wave band.
[8] A training apparatus according to an embodiment of the present invention is a training apparatus for neurofeedback training and includes an acquisition unit configured to acquire an electroencephalogram signal at a site in a left hemisphere of a subject and a control unit configured to output first teaching information for affecting an electroencephalogram signal by voice and then output second teaching information for affecting an electroencephalogram signal by voice. The control unit outputs, by voice, or causes the display device to display result information generated based on at least one of an electroencephalogram signal received from the acquisition unit for a predetermined time from a start of voice output of the first teaching information or an electroencephalogram signal received from the acquisition unit for a predetermined time from a start of voice output of the second teaching information.
[9] A method according to an embodiment of the present invention is a method for neurofeedback training and includes causing a display device to display first teaching information for affecting an electroencephalogram signal for a predetermined time, causing the display device to display second teaching information for affecting an electroencephalogram signal for a predetermined time after displaying the first teaching information, and causing the display device to display result information generated based on at least one of an electroencephalogram signal at a site in a left hemisphere of a subject acquired while displaying the first teaching information or an electroencephalogram signal at the site in the left hemisphere of the subject acquired while displaying the second teaching information.
[10] A method according to an embodiment of the present invention is a method for neurofeedback training and includes outputting first teaching information for affecting an electroencephalogram signal by voice, outputting second teaching information for affecting an electroencephalogram signal after the step of outputting first teaching information by voice, and outputting, by voice, or causing a display device to display result information generated based on at least one of an electroencephalogram signal at a site in the left hemisphere of a subject acquired for a predetermined time from a start of voice output of the first teaching information or an electroencephalogram signal at the site in the left hemisphere of the subject acquired for a predetermined time after a start of voice output of the second teaching information.
[11] A program according to an embodiment of the present invention causes a computer to execute a method described in [9] or [10].

### Advantageous Effects of Invention

According to the present invention, neurofeedback training can be performed (or supported) more easily.

### BRIEF DESCRIPTION OF DRAWINGS

FIG. 1 is a schematic configuration diagram of a training apparatus according to an embodiment of the present invention.

FIG. 2 is a diagram illustrating positions at which electrodes of a brain wave measuring apparatus are placed in electrode placement in compliance with the International 10-20 System according to an embodiment of the present invention.

FIG. 3 is a block diagram illustrating a hardware configuration of an electronic apparatus according to an embodiment of the present invention.

FIG. 4 is a functional block diagram of the training apparatus according to an embodiment of the present invention.

FIG. 5 is a diagram illustrating an example of a flowchart of information processing for neurofeedback training by a control unit according to an embodiment of the present invention.

FIG. 6 illustrates an example of result information displayed on a display device by the control unit.

FIG. 7 is a diagram illustrating an overview of a flow of unit training of one round in Experiment 1.

FIG. 8 is a diagram illustrating an overview of a flow of unit training of one round in Experiment 2.

FIG. 9 is a diagram illustrating the activity of the left DLPFC observed through fMRI in Experiment 2.

FIG. 10 is a diagram illustrating the activity of the left DLPFC observed through fMRI in Experiment 2.

### DESCRIPTION OF EMBODIMENTS

Hereinafter, a training apparatus 1 according to an embodiment of the present invention will be described with reference to the drawings. Neurofeedback (a neurofeedback method) is a method of learning how to regulate one's own brain activity while monitoring features of the brain activity in real time and receiving feedback as to whether they are close to an ideal brain state. In addition, neurofeedback training is training using neurofeedback performed by a subject who wants to learn how to regulate brain activity. Abnormalities in activation of the left dorsolateral prefrontal cortex (DLPFC) and balance of the posterior cingulate cortex (PCC)/the precuneus are observed in patients with depression when performing verbal fluency tasks, which are cognitive tasks of activating the executive function (one cognitive function). Ruminative thinking has been suggested to be associated with hyperactivity of the PCC, and there are reports that the left DLPFC contributes to suppression of hyperactivity of the PCC. Neurofeedback targeting the left DLPFC or PCC to alleviate depressive symptoms including ruminative thinking has been conducted in the related art, and the activity of the left DLPFC and PCC has been measured by using fMRI. Further, depressive symptoms include symptoms of depression, a declined attention control ability, and ruminative thinking, and these are symptoms observed not only in depression but also in an undiagnosed stage of depression.

The present inventors have found that activation of the left DLPFC was observed when it was attempted to increase the electroencephalogram power at the FC5 site and activation of the PCC was observed when it was attempted to decrease the electroencephalogram power by using neurofeedback targeting the brain waves at the FC5 site in the electrode placement according to the International 10-20 System. In addition, the present inventors have found that, by manipulating the electroencephalogram power acquired from the FC5 site by using neurofeedback, it can be expected to enhance the attention control ability (such as a decrease in ruminative thinking and an increase in attention concentration), whereby improvement of depressive symptoms can be expected. Furthermore, the present inventors have found that, when the electroencephalogram power at the FC5 site was manipulated by using neurofeedback, θ waves were able to be manipulated and thus were suitable as a target brain wave indicator.

Technical features of embodiments of the present invention are based on the above findings, and a training apparatus 1 according to embodiments of the present invention is an apparatus for neurofeedback training for enhancing the attention control ability of a subject, and thus the subject can perform the neurofeedback training by using the training apparatus 1. In the present specification, a person performing neurofeedback training is referred to as a subject. Further, in the present specification, more detailed description than necessary may be omitted for convenience of description. For example, detailed description about already well-known matter and redundant description about substantially the same configuration may be omitted.

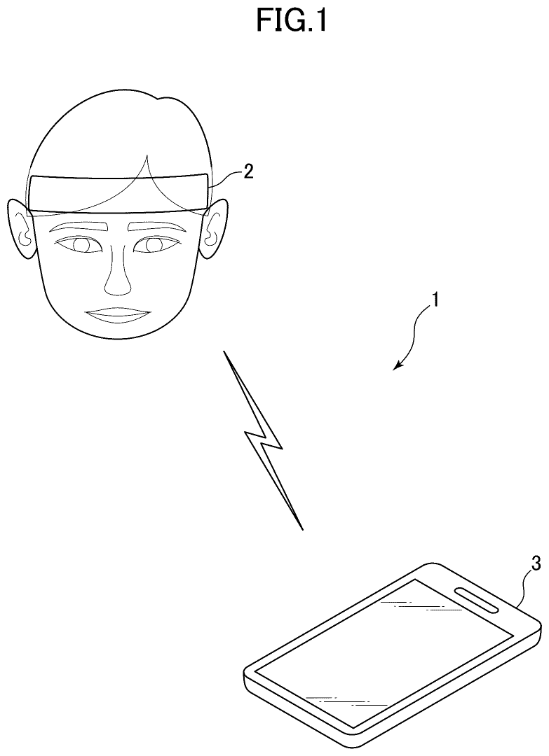

FIG. 1 is a schematic configuration diagram of a training apparatus 1 according to an embodiment of the present invention. The training apparatus 1 includes an electroencephalogram measuring apparatus 2 and an electronic apparatus 3.

The electroencephalogram measuring apparatus 2 includes one or more electrodes (not illustrated) and a communication unit (not illustrated) that transmits an electroencephalogram signal (electroencephalogram data) measured by the electrodes to the electronic apparatus 3. In the present embodiment, the electroencephalogram measuring apparatus 2 is a headgear-type (or headband-type) brain potential sensor (electroencephalograph) in which electrodes are placed to come into contact with a predetermined site of a subject when the subject is wearing the apparatus on his or her head as illustrated in FIG. 1. The electrodes are attached in contact with the head and measure (acquire) electroencephalogram signals based on brain activity.

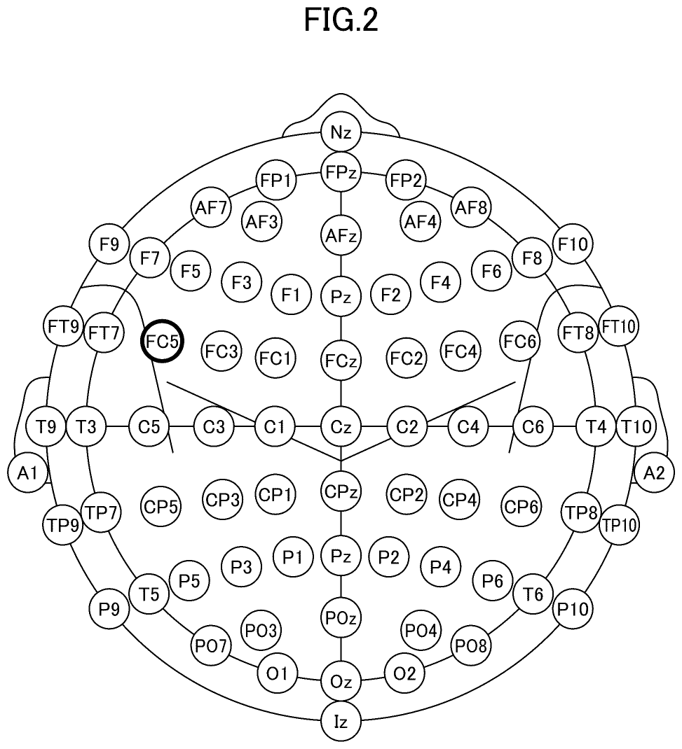

In the present embodiment, the electroencephalogram measuring apparatus 2 is configured such that, when a subject wears the apparatus, an electrode is placed at the FC5 site in the electrode placement according to the International 10-20 System as illustrated in FIG. 2 and acquires electroencephalogram signals from the site. In FIG. 2, the FC5 site is highlighted.

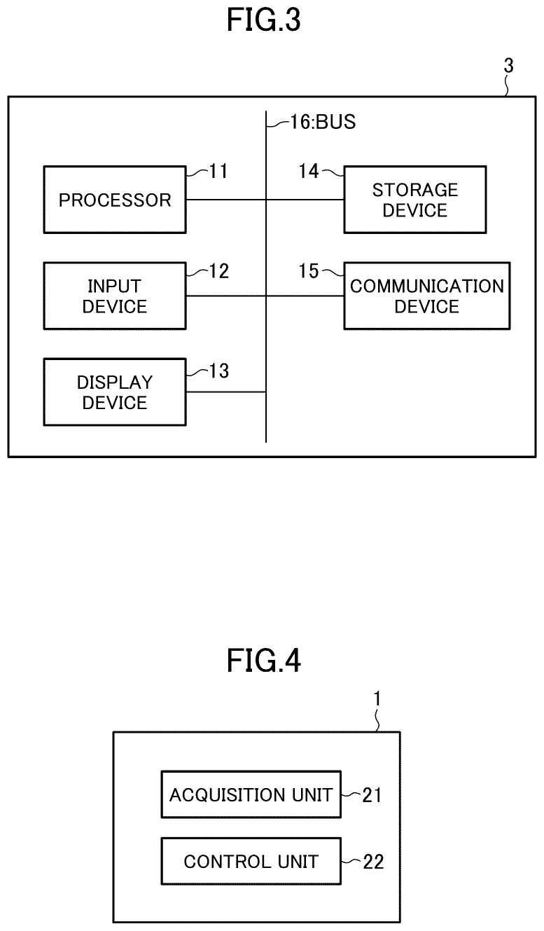

FIG. 3 is a block diagram illustrating a hardware configuration of the electronic apparatus 3 according to an embodiment of the present invention. The electronic apparatus 3 includes a processor 11, an input device 12, a display device 13, a storage device 14, and a communication device 15. These constituent devices are connected to each other by a bus 16. Further, an interface is assumed to be interposed between the bus 16 and each constituent device as necessary. The electronic apparatus 3 may include a configuration similar to that of a general server or personal computer (PC). For example, the electronic apparatus 3 is a smartphone or a tablet terminal.

The processor 11 controls all the operations of the electronic apparatus 3. For example, the processor 11 is a CPU. The processor 11 reads and executes programs and data stored in the storage device 14 to execute various kinds of processing. The processor 11 may include a plurality of processors.

The input device 12 is a user interface that receives input from a user to the electronic apparatus 3, and is, for example, a touch panel, a touch pad, a keyboard, a mouse, a button, or a sensor. The display device 13 is a display that displays an application screen or the like to the user of the electronic apparatus 3 under control of the processor 11. In one example, the input device 12 is a touch panel and has a structure in which the input device is integrated with the display device 13 (display). In one example, the electronic apparatus 3 includes, in addition to the display device 13, a voice output device that emits voice under control of the processor 11. For example, the voice output device is a speaker.

The storage device 14 includes a main storage device and an auxiliary storage device. The main storage device is, for example, a semiconductor memory such as a RAM. The RAM is a volatile storage medium capable of reading and writing information at a high speed and is used as a storage area and a work area when the processor 11 processes information. The main storage device may include a ROM, which is a read-only nonvolatile storage medium. The auxiliary storage device stores various programs and data used by the processor 11 when executing those programs. The auxiliary storage device may be any nonvolatile storage or nonvolatile memory as long as it can store information and may be detachable.

The communication device 15 exchanges data with another computer such as a user terminal or a server via a network, and is, for example, a wireless LAN module. The communication device 15 can be another wireless communication device or module such as a Bluetooth (trade name) module or can be a wired communication device or module such as an Ethernet (trade name) module or a USB interface.

The electroencephalogram measuring apparatus 2 transmits an electroencephalogram signal acquired by the electrodes to the electronic apparatus 3, and the electronic apparatus 3 acquires the electroencephalogram signal via the communication device 15.

FIG. 4 is a functional block diagram of the training apparatus 1 according to an embodiment of the present invention. The training apparatus 1 includes an acquisition unit 21 and a control unit 22. Since these functions are implemented by the processor 11 executing programs, for example, one part (function) may be partly or entirely included by another part. However, these functions may be implemented also by hardware by configuring an electronic circuit or the like for implementing some or all of the functions. The training apparatus 1 can also have other functions.

The acquisition unit 21 acquires, via the electrodes of the electroencephalogram measuring apparatus 2, an electroencephalogram signal in the frequency band of θ waves (4 to 7 Hz) at the FC5 site of the subject. The acquisition unit 21 transmits the electroencephalogram signal in the frequency band of the θ wave to the control unit 22 (electronic apparatus 3).

The control unit 22 executes processing for neurofeedback training and causes the display device 13 to display teaching information or the like for affecting the electroencephalogram signal acquired by the acquisition unit 21. The control unit 22 receives the electroencephalogram signal from the acquisition unit 21. In addition, the control unit 22 receives an input from the user to the electronic apparatus 3 via the input device 12. In one example, a dedicated application for executing information processing for neurofeedback training is installed in the training apparatus 1, and the control unit 22 is implemented through operations of the application. The control unit 22 causes the display device 13 to display a screen of the application. The control unit 22 stores the calculated or generated data in a predetermined memory area in the storage device 14 as necessary.

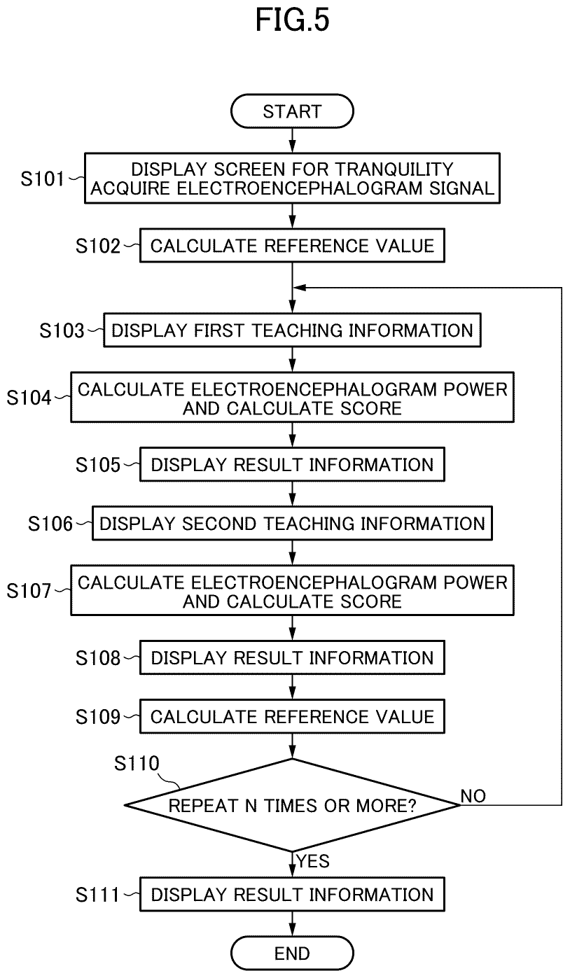

FIG. 5 is a diagram illustrating a flowchart of information processing for neurofeedback training of the control unit 22 according to an embodiment of the present invention. The flowchart illustrated in FIG. 5 illustrates information processing for neurofeedback training (unit training) for one session, the control unit 22 executes the information processing, and thereby the subject can perform unit training for one session.

In step 101, the control unit 22 causes the display device 13 to display information for resting for a predetermined time T1. In one example, the predetermined time T1 is thirty seconds. The information for resting is information presented to the subject in order to acquire an electroencephalogram signal of the subject in resting-state, and in one example, the information for resting is a vision fixation point (cross mark) displayed on the screen of the display device 13.

In step 102, the control unit 22 calculates a power (electroencephalogram power) for each predetermined time τ2 from the electroencephalogram signal received from the acquisition unit 21 while the information for resting is displayed in step 101. The control unit 22 calculates reference values including a baseline reference value and a normalized reference value from multiple calculated electroencephalogram powers. For example, the power for each predetermined time τ2 is an average power in each slot of the predetermined time τ2. In one example, the predetermined time T1 is thirty seconds, and the predetermined time τ2 is one second. In this case, the control unit 22 calculates thirty electroencephalogram powers for each slot of one second. In one example, the baseline reference value is an average value of multiple electroencephalogram powers calculated by the control unit 22 for each predetermined time τ2, and the normalized reference value is the difference between the maximum value and the minimum value of the multiple electroencephalogram powers calculated by the control unit 22 for each predetermined time τ2.

In step 103, the control unit 22 causes the display device 13 to display first teaching information for affecting the electroencephalogram signal for a predetermined time T3. In the present embodiment, the first teaching information is information for prompting an increase in the electroencephalogram power of the FC5 site of the subject, that is, information for prompting an increase in the intensity of the electroencephalogram signal acquired by the acquisition unit 21. In one example, the predetermined time T3 is thirty seconds. In one example, the first teaching information is an orange upward arrow displayed on the screen of the display device 13.

In step 104, the control unit 22 calculates the electroencephalogram power for each predetermined time τ4 from the electroencephalogram signal received from the acquisition unit 21 while the first teaching information is displayed. The control unit 22 calculates the average value of the multiple calculated electroencephalogram powers, and calculates the evaluation score (first evaluation score) for evaluating a change in the electroencephalogram power of the subject to whom the first teaching information is presented based on the calculated average value and the reference value calculated in step 102. For example, the power for each predetermined time τ4 is an average power in each slot of the predetermined time τ4. In one example, the predetermined time T3 is thirty seconds, and the predetermined time τ4 is one second. In this case, the control unit 22 calculates thirty electroencephalogram powers for each slot of one second. In one example, the control unit 22 calculates the difference between the calculated average value and the baseline reference value, and calculates the degree of the calculated value with respect to the normalized reference value as a first evaluation score.

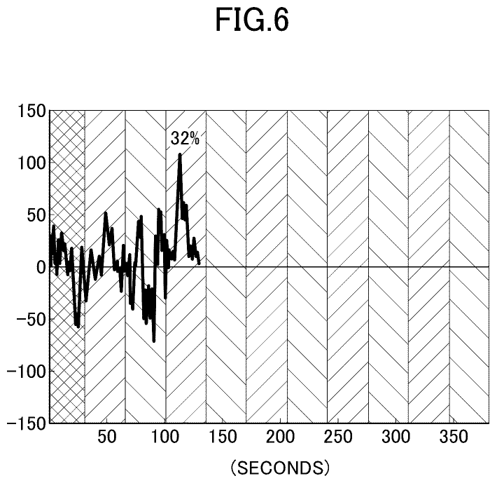

In step 105, the control unit 22 generates result information (first result information) indicating the first evaluation score calculated in the previous step 104, and causes the display device 13 to display the result information for a predetermined time T5. The first result information includes the first evaluation score calculated in the previous step 104 together with information chronologically indicating the electroencephalogram power for each τ4 calculated in previous step 104. In one example, the predetermined time T5 is five seconds. FIG. 6 is an example of the first result information displayed on the display device 13. In FIG. 6, “32%” is the first evaluation score.

In step 106, the control unit 22 causes the display device 13 to display second teaching information for affecting the electroencephalogram signal for a predetermined time T6. In the present embodiment, the second teaching information is information for prompting a decrease in the electroencephalogram power at the FC5 site of the subject, that is, information for prompting a decrease in the intensity of the electroencephalogram signal acquired by the acquisition unit 21. In this way, the control unit 22 displays the first teaching information for the predetermined time T3, and then displays the second teaching information for the predetermined time T6. The predetermined time T6 is the same time as the predetermined time T3, and in one example, the predetermined time T6 is thirty seconds. In one example, the first teaching information is a blue downward arrow displayed on the screen of the display device 13.

In step 107, the control unit 22 calculates the electroencephalogram power for each predetermined time τ7 from the electroencephalogram signal received from the acquisition unit 21 while the second teaching information is displayed. The control unit 22 calculates the average value of the multiple calculated electroencephalogram powers, and calculates the evaluation score (second evaluation score) for evaluating a change in the brain waves of the subject to whom the second teaching information is presented based on the calculated average value and the reference value calculated in step 102. For example, the power for each predetermined time τ7 is an average power in each slot of the predetermined time τ7. In one example, the predetermined time T6 is thirty seconds, and the predetermined time τ7 is one second. In this case, the control unit 22 calculates thirty electroencephalogram powers for each slot of one second. In one example, the control unit 22 calculates the difference between the calculated average value and the baseline reference value, and calculates the degree of the calculated value with respect to the normalized reference value as a second evaluation score.

In step 108, the control unit 22 generates result information (second result information) indicating the second evaluation score calculated in the previous step 107, and causes the display device 13 to display the result information for a predetermined time T8. The second result information includes the second evaluation score calculated in the previous step 107 together with information chronologically indicating the electroencephalogram power for each τ7 calculated in previous step 107. In one example, the predetermined time T8 is five seconds.

In step 109, the control unit 22 calculates the reference value including the baseline reference value and the normalized reference value from the plurality of electroencephalogram powers calculated in step 104 and the plurality of electroencephalogram powers calculated in step 107 (electroencephalogram powers of reference value calculation targets) in the previous steps 104 to 108. The control unit 22 updates the reference value calculated in step 102 with the reference value calculated in step 109. In one example, the baseline reference value is the average value of the electroencephalogram powers of the reference value calculation target, and the normalized reference value is the difference between the maximum value and the minimum value of the electroencephalogram powers of the reference value calculation target. In one example, when the predetermined times T3 and T6 are thirty seconds and the predetermined times τ4 and τ7 are one second, the control unit 22 calculates the reference value from a total of 60 electroencephalogram powers including 30 electroencephalogram powers calculated for 30 seconds of the predetermined time T3 and 30 electroencephalogram powers calculated for 30 seconds of the predetermined time T6.

In step 110, the control unit 22 determines whether steps 103 to 109 have been repeated a predetermined number of times (N times) or more. When it is determined in step 110 that the above steps have been repeated the predetermined number of times or more, the flowchart proceeds to step 111, and when it is determined that the above steps have been repeated less than the predetermined number of times, the flowchart proceeds to step 103, and the control unit 22 executes steps 103 to 109. In this way, the control unit 22 is configured to execute steps 103 to 109 the predetermined number of times. That is, the control unit 22 is configured to execute display of the first teaching information, display of the first result information, display of the second teaching information, and display of the second result information in this order the predetermined number of times. In one example, the predetermined number of times is five.

In step 104 of the second and following rounds, the control unit 22 calculates the average value of the multiple calculated electroencephalogram powers, and calculates the first evaluation score for evaluating a change in the electroencephalogram power of the subject to whom the first teaching information has been presented based on the calculated average value and the reference value updated in step 109. Likewise in step 107 of the second and following rounds, the control unit 22 calculates the average value of the multiple calculated electroencephalogram powers, and calculates the second evaluation score for evaluating a change in the electroencephalogram power of the subject to whom the second teaching information has been presented based on the calculated average value and the reference value updated in step 109.

In step 109 of the second and following rounds, the control unit 22 updates the reference value calculated in the previous step 109 with the reference value calculated in the current step 109.

In step 111, the control unit 22 calculates the total score based on the first evaluation score calculated in step 104 and the second evaluation score calculated in step 107, generates overall result information indicating the calculated total score, and causes the display device 13 to display the overall result information. In one example, the control unit 22 calculates, as the total score, the difference between the average value of the first evaluation scores calculated in step 104 of each round and the average value of the second evaluation scores calculated in step 107 of each round or a value corresponding to the difference.

When the control unit 22 executes steps 101 to 111 while the subject is wearing the electroencephalogram measuring apparatus 2, the subject is presented with a screen for resting for the predetermined time T1, then presented with the first teaching information for the predetermined time T3, the first result information for the predetermined time T5, the second teaching information for the predetermined time T6, and the second result information for the predetermined time T8 in this order a predetermined number of times, and then presented with the overall result information. As a result, the subject can complete unit training for one session. In one example, when performing neurofeedback training, the subject performs three sessions of unit training per day for several days (e.g., five days). As described above, in the neurofeedback training of the present embodiment, in order to enhance the function of switching attention in the two states in which the electroencephalogram power of the FC5 site is increased/decreased, the control unit 22 executes a process of alternately switching between presentation of the first teaching information and presentation of the second teaching information. In addition, in order to effectively perform neurofeedback training for enhancing the attention control ability, the control unit 22 presents the first teaching information/the second teaching information, and then performs, apart from this presentation, information processing of providing a feedback period for presenting the first result information/the second result information.

In one example, before performing the neurofeedback training, for example, before performing step 101, the control unit 22 causes the display device 13 to display an explanation screen for explaining the overview of the unit training for one session to the subject.

In one example, before performing neurofeedback training on the subject, for example, before performing step 101, the control unit 22 causes the display device 13 to display first hint information for increasing the electroencephalogram power of the subject at the FC5 and second hint information for decreasing the electroencephalogram power of the subject at the FC5 site. The first hint information includes, for example, a message “it is said that activity will be boosted when the subject is concentrating on a cognitive activity, such as recollecting past memories, performing a calculation, playing a word chain game, or imagining himself/herself singing”. The second hint information includes, for example, a message “it is said that activity will be reduced when the subject is concentrating on a sense of the body for breathing, looking blurry and broadly, concentrating on the pulse of a hand, a foot, a heart, or the like, emptying his/her head just as before going to sleep, doing nothing, or the like”. In another example, the control unit 22 can also display the first hint information together with the first teaching information and display the second hint information together with the second teaching information.

Next, neurofeedback training for enhancing an attention control ability performed by using the training apparatus 1 according to the embodiment of the present invention will be described based on the following Experiment 1. In order to confirm the effects of Experiment 1, neurofeedback training was performed by using the training apparatus 1, and at the same time, brain activity states of the left DLPFC and PCC were observed through fMRI.

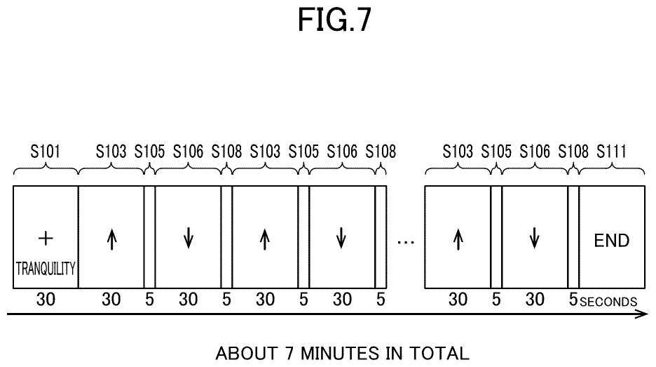

In Experiment 1, data of six healthy subjects for a total of nine rounds was acquired (two subjects participated multiple times). In Experiment 1, the subjects wearing the electroencephalogram measuring apparatus 2 performed neurofeedback training by using the control unit 22 performing information processing for the training illustrated in FIG. 5 on electroencephalogram signals of the θ wave acquired from the FC5 site. In addition, in Experiment 1, the predetermined times T1, T3, and T6 were all 30 seconds, the predetermined times T5 and T8 were all 5 seconds, the predetermined times τ2, τ4, and τ7 were all one second, the predetermined number of times N set in step 110 was five, and the duration of unit training of one round was about 7 minutes. In addition, in Experiment 1, the information for resting was a fixation point (cross mark) displayed on the screen of the display device 13, the first teaching information was an upward orange arrow displayed on the screen of the display device 13, and the second teaching information was a downward blue arrow displayed on the screen of the display device 13. In addition, in Experiment 1, each subject performed unit training three times a day for five days. FIG. 7 is a diagram illustrating an overview of a flow of the unit training of one round (one session) in Experiment 1.

As a result of Experiment 1, activation of the left DLPFC when an attempt was made to increase the electroencephalogram power at the FC5 site was observed, and activation of the PCC when an attempt was made to decrease the electroencephalogram power was observed. Therefore, it is thought that neurofeedback for enhancing an attention control ability can be performed by the training apparatus 1.

In one example, in step 101, the control unit 22 can output the information for resting by voice instead of or in addition to the information for resting displayed on the display device 13. In this case, for example, the information for resting to be output by voice is a voice output “Measurement will be started now. Please close your eyes” at the start of step 101. In one example, in step 103, the control unit 22 can output the first teaching information by voice without causing or in addition to causing the display device 13 to display the first teaching information. In this case, for example, the first teaching information to be output by voice is a voice output “Training of increasing the electroencephalogram power will be started now. Please start” at the start of step 103, and can also include a voice output “Please open your eyes” at the end of step 103. In one example, in step 105, the control unit 22 can output the first result information by voice without causing or in addition to causing the display device 13 to display the first result information. In one example, in step 106, the control unit 22 can output the second teaching information by voice without causing or in addition to causing the display device 13 to display the second teaching information. In this case, for example, the second teaching information to be output by voice is a voice output “Training of decreasing the electroencephalogram power will be started now. Please close your eyes. Please start.” at the start of step 106, and can also include a voice output “Please open your eyes” at the end of step 106. In one example, in step 108, the control unit 22 can output the second result information by voice without causing or in addition to causing the display device 13 to display the second result information. In one example, in step 111, the control unit 22 can output the overall result information by voice without causing or in addition to causing the display device 13 to display the overall result information.

Neurofeedback training for enhancing an attention control ability performed by using the training apparatus 1 according to the embodiment of the present invention will be described based on the following Experiment 2. In order to confirm the effects of Experiment 2, neurofeedback training was performed by using the training apparatus 1, and at the same time, brain activity states of the left DLPFC and PCC were observed through fMRI.

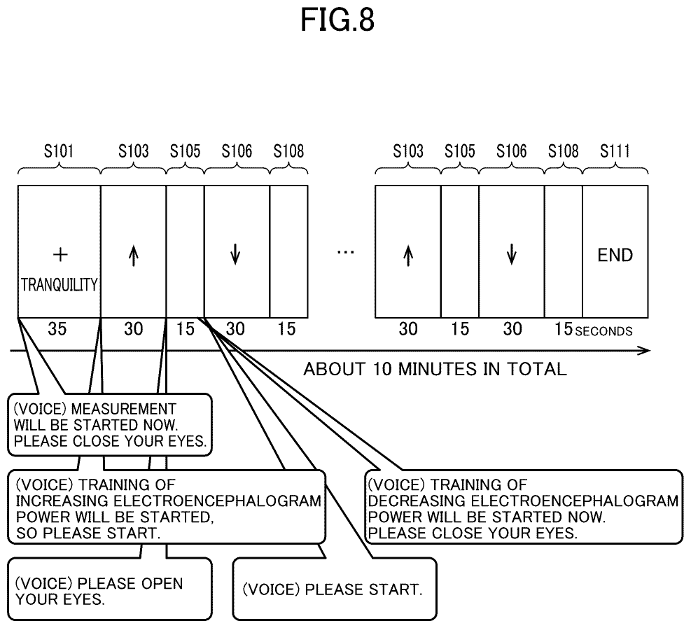

In Experiment 2, data was acquired from ten healthy subjects. In Experiment 2, the subjects wearing the electroencephalogram measuring apparatus 2 performed neurofeedback training by using the control unit 22 performing information processing for the training illustrated in FIG. 5 on electroencephalogram signals of the θ wave acquired from the FC5 site. In addition, in Experiment 2, the predetermined times T1 was 35 seconds, the predetermined times T3 and T6 were all 30 seconds, the predetermined times T5 and T8 were all 15 seconds, the predetermined times τ2, τ4, and τ7 were all one second, the predetermined number of times N set in step 110 was five, and the duration of unit training of one round was about 10 minutes. In addition, in Experiment 2, the information for resting was the fixation point (cross mark) displayed on the screen of the display device 13 and a voice guide output from the speaker (a voice output “Measurement will be started now. Please close your eyes” at the start of the presentation of the information for resting), the first teaching information was an orange upward arrow displayed on the screen of the display device 13 and a voice guide output from the speaker (a voice output “Training of increasing the electroencephalogram power will be started now. Please start” at the start of the presentation of the first teaching information, and a voice output “Please open your eyes” at the end of the presentation of the first teaching information), and the second teaching information was a blue downward arrow displayed on the screen of the display device 13 and a voice guide output from the speaker (voice outputs “Training of decreasing the electroencephalogram power will be started now. Please close your eyes” and “Please start” at the start of the presentation of the second teaching information). In addition, in Experiment 2, each subject performed unit training three times a day for five days. FIG. 8 is a diagram illustrating an overview of a flow of the unit training of one round (one session) in Experiment 2.

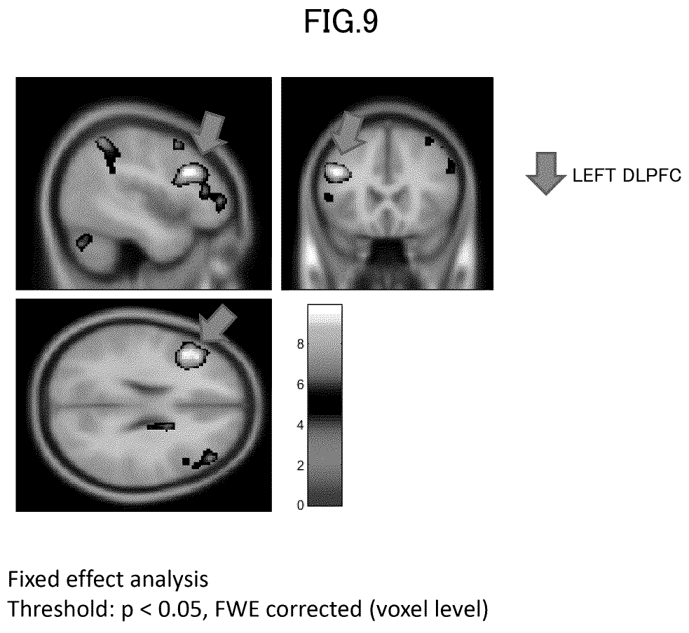

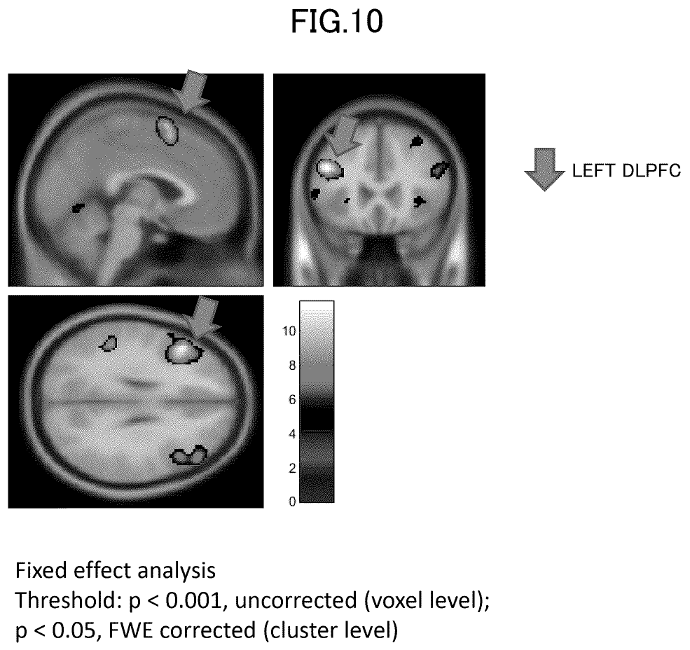

As a result of Experiment 2, the activation of the left DLPFC was observed when the electroencephalogram power at the FC5 site was intended to be increased, and by repeating the training for five days, the activity of the left DLPFC was significantly increased on the fifth day compared to the first day as illustrated in FIG. 9. In addition, when analysis was performed based on the difference in activity between when the electroencephalogram power at the FC5 site was intended to be increased and when the electroencephalogram power at the site was intended to be decreased, activation of the left DLPFC was observed, and by repeating the training for five days, the activity of the left DLPFC significantly increased on the fifth day compared to the first day as illustrated in FIG. 10. Therefore, it is thought that neurofeedback for enhancing an attention control ability can be performed by the training apparatus 1.

Next, operations and effects of the training apparatus 1 according to the embodiment of the present invention will be described.

Since the past, fMRI has been used when performing neurofeedback for alleviating depressive symptoms because it was necessary to observe the brain activity of the left DLPFC and PCC. However, there is time constraints on many medical institutions using MRI, and places other than medical institutions where MRI is installed are limited, and thus there is a problem that an environment used for training of fMRI neurofeedback is not prepared.

As described above, the present inventors have found that brain waves at the FC5 site are correlated with the brain activity of the left DLPFC and the PCC, and also found that neurofeedback training for increasing the attention control capabilities can be performed by repeatedly prompting subjects to increase/decrease the electroencephalogram power of the θ wave and presenting the analysis result to the subjects.

In the present embodiment, the acquisition unit 21 acquires the electroencephalogram signals at the FC5 site of the subjects in the θ wave band, and the control unit 22 executes the information processing for the neurofeedback training, and thereby the subjects are able to perform the neurofeedback training. To be more specific, the neurofeedback training of the present embodiment is configured to alternately switch between the “high attention concentration state (the left DLPFC in high activity/PCC in low activity)” time (step 103, for example, 30 seconds) for increasing the electroencephalogram power at the FC5 site and the “low attention concentration state (left DLPFC in low activity/PCC in high activity)” time (step 106, for example, 30 seconds) for decreasing the electroencephalogram power, and thereby the training for improving the function of switching the attention between the above-described two states can be conducted. In addition, the feedback periods (step 105/step 108, for example, 5 seconds) for presenting the first result information/second result information are separately set after the time for increasing/decreasing the electroencephalogram power, the “period for concentrating on self-control” and the “period for concentrating on confirmation of the results are configured to be separated, and thereby neurofeedback training for further enhancing the attention control ability can be conducted. In addition, since the processing (step 111) of presenting the overall result information is executed at the end of the unit training, it is possible to give motivation to the training of the subjects.

Since measurement of brain waves is inexpensive and easy as compared with MRI, neurofeedback training for enhancing the attention control function can be more easily performed or supported by adopting the configuration of the present embodiment. In addition, effects on clinical psychological states such as ruminative thinking, depressive symptoms, cognitive functions, and attention functions can be expected through neurofeedback training for enhancing the attention control function. Further, these symptoms can also be collectively referred to as “depressive symptoms”. Examples of known indices for measuring the effects of neurofeedback training on these symptoms include the reflection scale and the ruminating response scale for ruminative thinking, the Beck's depression questionnaire and mental and physical questionnaire for depressive symptoms, the 2-back task and CANTAB for cognitive functions, and the daily attentional experience questionnaire for attentional functions.

In addition, in the present embodiment, the control unit 22 uses a reference value that is updated each time steps 103 to 108 are performed when calculating the evaluation score, thereby updating the reference value that can change in accordance with the state in which the subjects are trying to increase/decrease the electroencephalogram power, and reducing the possibility of overestimating or underestimating the change in the electroencephalogram power.

The above operations and effects are the same in other embodiments and other examples unless otherwise specified.

In the embodiment of the present invention, it is possible to perform neurofeedback training for enhancing the attention control function by adjusting various parameters and performing information processing for neurofeedback training even under conditions different from those in the preliminary experiment.

In the embodiment of the present invention, the control unit 22 can be configured to perform information processing according to a flowchart different from the flowchart illustrated in FIG. 5 within a range in which neurofeedback training for enhancing the attention control ability can be conducted.

In one or more embodiments of the present invention, after causing the display device 13 to display the overall result information in step 111, the control unit 22 can present encouragement or recommendation to the subjects through display on the display device 13 or a voice output from a voice output device (for example, a speaker). For example, the control unit 22 can present the subjects with voice or text saying “The current session was successful. Keep it up for the next session”, “Try a different strategy in the next session”, “There are some people who can increase the brain waves well when doing math in their heads”, or the like emitted from a voice output device (for example, a speaker) or displayed on the display device 13. By configuring to present encouragement or recommendation to the subjects in this way, it is possible to enhance the attention control abilities of the subjects, for example, to enhance learning efficiency.

In one or more embodiments of the present invention, the electronic device 3 may include a voice output device (for example, a speaker) that emits voice according to control of the processor 11, and may not include the display device 13. In one example, the control unit 22 can output the information for resting by voice (for example, the voice “Measurement will be started now. Please close your eyes”) in step 101, and after the voice output of the information for resting ends (for example, after the predetermined time T1 elapses from the start of the voice output), the first teaching information can be output by voice in step 103. In this case, for example, the control unit 22 calculates, in step 102, the power (electroencephalogram power) for each predetermined time τ2 from the electroencephalogram signal received from the acquisition unit 21 for the predetermined time T1 from the start of the voice output of the information for resting in step 101 (until the predetermined time T1 elapses from the start of the voice output of the information for resting), and the control unit 22 can calculate the reference values including the baseline reference value and the normalized reference value from the multiple calculated electroencephalogram powers. In one example, the control unit 22 can output the first teaching information by voice (for example, the voice “Training of increasing the electroencephalogram power will be started now. Please start”) in step 103, and after the voice output of the first teaching information ends (for example, after the predetermined time T3 elapses from the start of the voice output), the first result information can be output by voice in step 105. In this case, for example, the control unit 22 calculates, in step 104, the electroencephalogram power for each predetermined time τ4 from the electroencephalogram signal received from the acquisition unit 21 for the predetermined time T3 from the start of the voice output of the first teaching information in step 103 (until the predetermined time T3 elapses from the start of the voice output of the first teaching information), and then the control unit 22 can calculate the average value of the multiple calculated electroencephalogram powers, and calculate the evaluation score (first evaluation score) for evaluating the change in the electroencephalogram power of the subject presented with the first teaching information based on the calculated average value and the reference value calculated in step 102 or 109. In one example, the control unit 22 can output the first result information by voice in step 105, and can output the second teaching information by voice in step 106 after the voice output of the first result information ends (for example, after the predetermined time T5 elapses from the start of the voice output). In one example, the control unit 22 can output the second teaching information by voice (for example, the voice “Training of decreasing the electroencephalogram power will be started now. Please close your eyes. Please start”) in step 106, and after the voice output of the second teaching information ends (for example, after the predetermined time T6 elapses from the start of the voice output), the second result information can be output by voice in step 108. In this case, for example, the control unit 22 calculates, in step 107, the electroencephalogram power for each predetermined time τ7 from the electroencephalogram signal received from the acquisition unit 21 for the predetermined time T6 from the start of the voice output of the second teaching information in step 106 (until the predetermined time T6 elapses from the start of the voice output of the second teaching information), and then the control unit 22 can calculate the average value of the multiple calculated electroencephalogram powers, and calculate the evaluation score (second evaluation score) for evaluating the change in the electroencephalogram power of the subject presented with the second teaching information based on the calculated average value and the reference value calculated in step 102 or 109. In one example, the control unit 22 can output the second result information by voice in step 108, and can output the first teaching information by voice in step 103 or can output the overall result information by voice in step 111 after the voice output of the second result information ends (for example, after the predetermined time T8 elapses from the start of the voice output). Further, in the above example, “after the voice output ends” may mean a time immediately after the end of the voice output or a time after a predetermined time elapses from the end of the voice output.

In another embodiment of the present invention, a program for implementing the functions of the embodiment of the present invention described above and the information processing illustrated in the flowcharts and a computer-readable storage medium storing the program can be provided. In addition, in another embodiment, a method for implementing the functions of the embodiment of the present invention described above and the information processing illustrated in the flowcharts can be provided. In addition, in another embodiment, a server that can supply a computer with the program for implementing the functions of the embodiment of the present invention described above and the information processing illustrated in the flowcharts can be provided. In addition, in another embodiment, a virtual machine that implements the functions of the embodiment of the present invention described above and the information processing illustrated in the flowcharts can be provided.

In the embodiment of the present invention, an electroencephalogram signal received from the acquisition unit 21 while the control unit 22 displays predetermined information may mean an electroencephalogram signal acquired by the acquisition unit 21 while the control unit 22 displays the predetermined information.

In one or more embodiments of the present invention, the contents of the first teaching information and the second teaching information can be reversed within a range in which neurofeedback training for enhancing the attention control ability can be conducted.

In one or more embodiments of the present invention, the control unit 22 can be configured to display the first result information in step 105 only when the first evaluation score is equal to or greater than a predetermined threshold, less than the threshold, or within a predetermined range within the range in which neurofeedback training for enhancing the attention control ability can be conducted. In one or more embodiments of the present invention, the control unit 22 can be configured to display the second result information in step 108 only when the second evaluation score is equal to or greater than a predetermined threshold, less than the threshold, or within a predetermined range within the range in which neurofeedback training for enhancing the attention control ability can be conducted. In one or more embodiments of the present invention, the control unit 22 can be configured to display the first result information and the second result information at different timings from step 105 and step 108 within the range in which neurofeedback training for enhancing the attention control ability can be conducted.

In one or more embodiments of the present invention, the control unit 22 may not use a reference value, may use only one of a baseline reference value and a normalized reference value, or may use another reference value in information processing for neurofeedback training within the range in which neurofeedback training for enhancing the attention control ability can be conducted. When the control unit 22 uses no reference values in the information processing, the flowchart may not include step 101, step 102, and step 109. In this case, the control unit 22 uses no reference values when calculating the evaluation score in step 104 and step 107.

In one or more embodiments of the present invention, the processing of step 102 may be performed partially or entirely simultaneously with step 101. In one or more embodiments of the present invention, the control unit 22 can perform the processing of step 104 partially or entirely simultaneously with step 103 or 105, can perform the processing of step 107 partially or entirely simultaneously with step 106 or 108, and can perform the processing of step 109 or step 110 partially or entirely simultaneously with step 108.

In one or more embodiments of the present invention, the control unit 22 can be configured not to perform step 111 and not to display the overall result information.

In one or more embodiments of the present invention, the acquisition unit 21 may be configured to transmit an acquired electroencephalogram signal to the control unit 22, instead of transmitting the electroencephalogram signal in the frequency band of the θ wave to the electronic apparatus 3, and the control unit 22 may be configured to extract the electroencephalogram signal in the θ wave band from the electroencephalogram signal received from the acquisition unit 21 and use the extracted electroencephalogram signal as an electroencephalogram signal. In addition, in this case, if neurofeedback training for enhancing the attention control ability can be conducted, the control unit 22 may use a signal including a part of the frequency band of the θ wave as an electroencephalogram signal in steps 102, 104, 107, and the like, or may use a signal including a band other than the frequency band of the θ wave as the electroencephalogram signal.

In one or more embodiments of the present invention, the electroencephalogram measuring apparatus 2 may not be a headgear-type (or headband-type) electroencephalograph, but may be a cap- or helmet-type electroencephalograph in which electrodes are placed in advance. Alternatively, the electroencephalogram measuring apparatus 2 can be an electroencephalograph in an arbitrary shape connected by wire from the electronic apparatus 3.

In one or more embodiments of the present invention, as long as neurofeedback training for enhancing the attention control ability can be conducted, the electroencephalogram measuring apparatus 2 may be configured such that the electrodes are placed at sites within predetermined distances from the FC5 site in the electrode placement according to the International 10-20 System and brain waves are acquired from the electrodes when it is being worn by a subject. For example, the sites within the predetermined distances from the FC5 site are sites within 5 mm, within 10 mm, within 15 mm, or within 20 mm from the FC5 site.

In one or more embodiments of the present invention, as long as neurofeedback training for enhancing the attention control ability can be conducted, the electroencephalogram measuring apparatus 2 may be configured such that the electrodes are placed at sites within the left frontal lobe or the left hemisphere within predetermined distances from the FC5 site in the electrode placement according to the International 10-20 System and brain waves are acquired from the electrodes when it is being worn by a subject.

In one or more embodiments of the present invention, when a subject has another disease and needs assistance of a third party when performing neurofeedback training, neurofeedback training may be performed via the third party. In this case, the third party can play a role of transmitting information to be displayed on the display device 13 to the subject. Alternatively, in one or more embodiments of the present invention, the training apparatus 1 may further include a transmission device for transmitting teaching information for a subject having another disease. In this case, the input device 12 may be a voice input device, a sensor that receives an input of gestures, or the like.

In one or more embodiments of the present invention, the training apparatus 1 can also be utilized as an apparatus capable of newly finding a thought, a recollection, an experience, a video, or the like for controlling the electroencephalogram power of a specific target at the FC5 site in the course of performing neurofeedback training with the subject. Specifically, it is possible to determine whether or not a new thought, recollection, experience, video, or the like controls the electroencephalogram power of the subject based on a specific thought, recollection, experience, video, or the like that has been found to control the electroencephalogram power of the subject with good reproducibility.

Although the neurofeedback training for depressive symptoms using the training apparatus 1 has been introduced in the embodiment of the present invention, in one or more embodiments of the present invention, neurofeedback using the training apparatus 1 can also be applied to anxiety, attention-deficit/hyperactivity disorder, behavioral disorders, sleep disorders, headaches and migraines, chronic pains, mood disorders such as depression and premenstrual dysphoric disorder, drug dependence, eating disorders, obsessive-compulsive disorder, epilepsy and seizures, autism spectrum disorders, stress-related disorder such as post-traumatic stress disorder, schizophrenic disorders, bipolar disorders, dementias, and the like. In addition, neurofeedback training using the training apparatus 1 can be applied to improve performance such as music or athletic competitions, which require mindfulness or high concentration.

In the processing or operations described above, the processing or operations can be modified freely as long as there is no occurrence of contradiction in the processing or operations such as using data that is not yet supposed to be used in a corresponding step. In addition, each example described above is exemplified for describing the present invention, and the present invention is not limited to these examples. The present invention may be implemented in various forms without departing from the scope thereof.

### REFERENCE SIGNS LIST

1 Training apparatus
2 Electroencephalogram measuring apparatus
3 Electronic apparatus
11 Processor
12 Input device
13 Display device
14 Storage device
15 Communication device
16 Bus
21 Acquisition unit
22 Control unit
23 Input unit
24 Display unit

1. A training apparatus for neurofeedback training, the training apparatus comprising:
an acquisition unit configured to acquire an electroencephalogram signal at a site in a left hemisphere of a subject; and
a control unit configured to cause a display device to display first teaching information for affecting an electroencephalogram signal for a predetermined time and to cause the display device to display second teaching information for affecting an electroencephalogram signal for a predetermined time after displaying the first teaching information,
wherein the control unit is configured to cause the display device to display result information generated based on at least one of an electroencephalogram signal received from the acquisition unit while displaying the first teaching information or an electroencephalogram signal received from the acquisition unit while displaying the second teaching information.

2. The training apparatus according to claim 1,
wherein the first teaching information is information for prompting an increase in an intensity of the electroencephalogram signal acquired by the acquisition unit, and
the second teaching information is information for prompting a decrease in the intensity of the electroencephalogram signal.

3. The training apparatus according to claim 1,
wherein the control unit is configured to:
cause the display device to display first result information generated based on the electroencephalogram signal received from the acquisition unit while displaying the first teaching information after displaying the first teaching information and
cause the display device to display second result information generated based on the electroencephalogram signal received from the acquisition unit while displaying the second teaching information after displaying the second teaching information, and
the control unit is configured to perform display of the first teaching information, display of the first result information, display of the second teaching information, and display of the second result information in this order a predetermined number of times.

4. The training apparatus according to claim 3,
wherein the control unit is configured to:
cause the display device to display information for resting for a predetermined time before displaying the first teaching information on the display device for training, determine a reference value based on the electroencephalogram signal received from the acquisition unit while the information for resting is being displayed, and
update the reference value based on the electroencephalogram signal received from the acquisition unit while the first teaching information is being displayed and the electroencephalogram signal received from the acquisition unit while the second teaching information is being displayed each time the display of the first teaching information, the display of the first result information, the display of the second teaching information, and the display of the second result information are performed in this order, and
the control unit is configured to generate the first result information based on the electroencephalogram signal received from the acquisition unit while the first teaching information is being displayed and the reference value determined or updated and generate the second result information based on the electroencephalogram signal received from the acquisition unit while the second teaching information is being displayed and the reference value determined or updated.

5. The training apparatus according to claim 3, wherein the control unit is configured to perform the display of the first teaching information, the display of the first result information, the display of the second teaching information, and the display of the second result information in this order a predetermined number of times and then cause result information based on the first result information and the second result information to be displayed.

6. The training apparatus according to claim 1, wherein the acquisition unit is configured to acquire an electroencephalogram signal at an FC5 site of a subject according to the International 10-20 System or a site within a predetermined distance from the site.

7. The training apparatus according to claim 1, wherein the electroencephalogram signal received from the acquisition unit is an electroencephalogram signal in a θ wave band.

8. A training apparatus for neurofeedback training, the training apparatus comprising:
an acquisition unit configured to acquire an electroencephalogram signal at a site in a left hemisphere of a subject; and
a control unit configured to output first teaching information for affecting an electroencephalogram signal by voice and then output second teaching information for affecting an electroencephalogram signal by voice,
wherein the control unit is configured to output, by voice, or cause the display device to display result information generated based on at least one of an electroencephalogram signal received from the acquisition unit for a predetermined time from a start of voice output of the first teaching information or an electroencephalogram signal received from the acquisition unit for a predetermined time from a start of voice output of the second teaching information.

9. A method for neurofeedback training, the method comprising:
causing a display device to display first teaching information for affecting an electroencephalogram signal for a predetermined time;
causing the display device to display second teaching information for affecting the electroencephalogram signal for a predetermined time after displaying the first teaching information; and
causing the display device to display result information generated based on at least one of an electroencephalogram signal at a site in a left hemisphere of a subject acquired while displaying the first teaching information or an electroencephalogram signal at the site in the left hemisphere of the subject acquired while displaying the second teaching information.

10. (canceled)

11. A non-transitory computer readable medium storing a program, wherein execution of the program causes a computer to execute a method described in claim 9.
